(19) United States
(12) Patent Application Publication
(10) Pub. No.: US 2019/0085390 A1
(43) Pub. Date: Mar. 21, 2019

(54) FLOW CELLS HAVING REACTIVE SURFACES FOR NUCLEIC ACID SEQUENCE ANALYSIS

(71) Applicant: Corning Incorporated, Corning, NY (US)

(72) Inventors: Ye Fang, Painted Post, NY (US); Jeffrey Glenn Lynn, Tioga, PA (US); Wei Sun, Painted Post, NY (US); Ying Wei, Painted Post, NY (US)

(21) Appl. No.: 16/127,617

(22) Filed: Sep. 11, 2018

Related U.S. Application Data
(60) Provisional application No. 62/559,951, filed on Sep. 18, 2017.

(51) Int. Cl. C12Q 1/6874

(52) U.S. Cl. CPC C12Q 1/6874

(57) ABSTRACT

A flow cell article including: a chamber; and at least one surface of the chamber comprising: a solid substrate having a reactive surface comprising: a coupling agent covalently attached to the solid substrate; a polymer of the formula (I) as defined herein, covalently attached to the coupling agent;

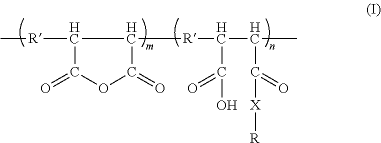

and

a nucleic acid probe covalently attached to the polymer. Also disclosed is a method of making the article and a method of using the article.

### CROSS-REFERENCE TO RELATED APPLICATIONS

The present application claims the benefit of priority under 35 U.S.C. § 119 to U.S. Provisional Patent Application No. 62/559,951, filed on Sep. 18, 2017, the contents of which are relied upon and incorporated herein by reference in its entirety.

The entire disclosure of each publication or patent document mentioned herein is incorporated by reference.

### BACKGROUND

The disclosure relates to a flow cells having a reactive surface for nucleic acid sequence analysis.

### SUMMARY

In embodiments, the disclosure provides a flow cell article having reactive surfaces for nucleic acid sequence analysis.

In embodiments, the disclosure provides a flow cell article having an amine-reactive polymeric coating for covalently coupling amine-terminated nucleic acid (e.g., DNA) probe molecules.

In embodiments, the disclosure provides a flow cell article having one or more amine-terminated nucleic acid (e.g., DNA) probe molecules covalently coupled to the amine-reactive polymeric coating.

In embodiments, the density of the attached nucleic acid (e.g., DNA) probe molecules can be precisely controlled.

In embodiments, the disclosure provides a method to control the density of amine-terminated nucleic acid (e.g., DNA) probe molecules attached, which can provide precise control of the hybridization amount of DNA fragments containing adaptor sequence(s) complementary to the nucleic acid probe, which can lead to improved polyclonal clustering and sequencing efficiency.

### BRIEF DESCRIPTION OF THE DRAWINGS

In embodiments of the disclosure:

FIG. 1 shows a schematic (100) of covalently coupled of DNA probe molecules to a solid support.

FIG. 2 shows a bar chart of the fluorescent intensity of a dA30-presenting surface after being hybridized with Cy3-labeled dT30 under different conditions.

FIG. 3 shows a bar chart of fluorescent intensity of a dA30-presenting surface after being treated with 0.05M NaOH and subsequently hybridized with Cy3-labeled dT30 under different conditions.

FIG. 4 shows a fluorescent image of a surface after being hybridized with Cy3-labeled dT30.

FIGS. 5A and 5B, respectively, show a photo image (5A) and fluorescent (5B) image of an entire flow cell having eight channels, each consisting of amine-terminated dA30 attached to a reactive polymer coating. The fluorescence (5B) image was obtained after being hybridization with Cy3-labeled dT30.

### DETAILED DESCRIPTION

Various embodiments of the disclosure will be described in detail with reference to drawings, if any. Reference to various embodiments does not limit the scope of the invention, which is limited only by the scope of the claims attached hereto. Additionally, any examples set forth in this specification are not limiting and merely set forth some of the many possible embodiments of the claimed invention.

In embodiments, the disclosed article and method of making and using provide one or more advantageous features or aspects, including for example as discussed below. Features or aspects recited in any of the claims are generally applicable to all facets of the invention. Any recited single or multiple feature or aspect in any one claim can be combined or permuted with any other recited feature or aspect in any other claim or claims.

### Definitions

“Next generation sequencing”, “NGS”, is a type of DNA sequencing technology that uses parallel sequencing of many small fragments of DNA from a biological sample to determine a gene sequence. NGS can be used to sequence every nucleotide in a genome, or small portions of the genome such as the exome or a preselected subset of genes.

“Glass” or like terms refers to glass and glass-ceramics that are suitable as substrates.

“Include,” “includes,” or like terms means encompassing but not limited to, that is, inclusive and not exclusive.

“About” modifying, for example, the quantity of an ingredient in a composition, concentrations, volumes, process temperature, process time, yields, flow rates, pressures, viscosities, and like values, and ranges thereof, or a dimension of a component, and like values, and ranges thereof, employed in describing the embodiments of the disclosure, refers to variation in the numerical quantity that can occur, for example: through typical measuring and handling procedures used for preparing materials, compositions, composites, concentrates, component parts, articles of manufacture, or use formulations; through inadvertent error in these procedures; through differences in the manufacture, source, or purity of starting materials or ingredients used to carry out the methods; and like considerations. The term “about” also encompasses amounts that differ due to aging of a composition or formulation with a particular initial concentration or mixture, and amounts that differ due to mixing or processing a composition or formulation with a particular initial concentration or mixture.

“Optional” or “optionally” means that the subsequently described event or circumstance can or cannot occur, and that the description includes instances where the event or circumstance occurs and instances where it does not.

The indefinite article “a” or “an” and its corresponding definite article “the” as used herein means at least one, or one or more, unless specified otherwise.

Abbreviations, which are well known to one of ordinary skill in the art, may be used (e.g., “h” or “hrs” for hour or hours, “g” or “gm” for gram(s), “mL” for milliliters, and “rt” for room temperature, “nm” for nanometers, and like abbreviations).

Specific and preferred values disclosed for components, ingredients, additives, dimensions, conditions, times, and like aspects, and ranges thereof, are for illustration only; they do not exclude other defined values or other values within defined ranges. The composition and methods of the disclosure can include any value or any combination of the values, specific values, more specific values, and preferred values described herein, including explicit or implicit intermediate values and ranges.

The embodiments, the present disclosure relates generally to nucleic acid analysis, and more specifically to methods and flow cell devices for use in, for example, massively parallel genomics analysis (e.g., next generation sequencing, NGS).

Many important molecular applications, such as DNA microarrays, NGS, or DNA based biosensors, use synthetic DNA probe molecules attached to solid supports including flat two-dimensional surfaces, such as glass, silica, or silicon slides, and to three-dimensional surfaces such as micro-beads and micro/nano-particles. The immobilization of DNA probe molecules on a surface can be achieved by numerous methods, for instance, electrostatic interaction, covalently coupling, entrapments, and like methods. The present disclosure provides materials and methods for covalently coupling of amine-terminated DNA probe molecules onto a solid support for nucleic acid analysis, in particular gene sequencing.

The past decades has seen extraordinary progress in cataloging human genetic variations, and correlating these variations with susceptibility to disease, responsiveness to specific therapies, susceptibility to dangerous drug side effects, and other medically actionable characteristics. Advances in NGS have led to a decreased cost per megabase and an increase in the number and diversity of genomes sequenced. Key to advances in genome wide sequencing is to use a flow cell to partition millions of DNA fragments generated from a biological DNA sample onto the surface of the flow cell so that almost all fragments that are immobilized can be sequenced simultaneously. Some of NGS technologies, in particular the short-read sequencing techniques, require the covalent immobilization of DNA fragments onto the surface of a flow cell for sequencing. Significant to sequencing efficiency and quality is the ability to achieve stable, reproducible, and optimal attachment of DNA molecules onto the surface of a flow cell.

The present disclosure provides a reactive surface to covalently capture amine-terminated DNA probe fragments. The fragment density and position can be precisely controlled, so that polyclonal clusters formed can be spatially controlled and sequenced efficiently with improved quality.

In embodiments, the disclosure provides a flow cell article comprising:
a chamber; and
at least one surface of the chamber comprising:
   a solid substrate, such as glass, having a reactive surface comprising:
      a coupling agent covalently attached to the solid substrate;
      a polymer of the formula (I) covalently attached to the coupling agent;

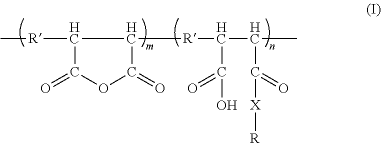

the polymer having at least one of: a plurality of maleic anhydride reactive groups (m), a plurality of reacted groups (n), or a mixture of (m) and (n), where
X can be, for example, a divalent NH, O, or S;
R can be, for example, H, a substituted or an unsubstituted, linear or branched alkyl group, an oligo(ethylene oxide), an oligo(ethylene glycol), or a dialkyl amine;
R′ can be, for example, a residue of a first unsaturated monomer that has been copolymerized with maleic anhydride;
the relative ratio (m:n) of the maleic anhydride reactive groups to the reacted groups is from 0.5 to 10;
m can be, for example, of from 1 to 10,000 and n can be, for example, of from 0 to 9,500; and
a nucleic acid probe covalently attached to the polymer.

In embodiments, the nucleic acid probe can be, for example, an amine-terminated nucleic acid or nucleic acid fragment.

In embodiments, the nucleic acid probe molecule can have a density, for example, of from 1 to 10,000 probe molecules to each of polymers of the formula (I).

In embodiments, the nucleic acid probe molecule can have a density of, for example, from 1 to 500,000 probe molecules per square micrometer of surface area. Preferably, the nucleic acid probe molecule can have a density of, for example, from 1,000 to 500,000 probe molecules per square micrometer (μm²) surface area when polyclonal clustering is required for sequencing. Alternatively, the nucleic acid probe molecule can have a density, for example, of from 1 to 1000 probe molecules per μm² surface area when single molecule analysis is called for, for sequencing.

In embodiments, the coupling agent can be, for example, an amine functionalized silane, silsesquioxane, or a mixture thereof.

In embodiments, the amine functionalized silane can be, for example, 3-(aminopropyl)triethoxysilane, and the silsesquioxane can be, for example, aminopropylsilsesquioxane.

In embodiments, the disclosure provides a method of making the abovementioned article, comprising:
contacting a solid substrate with a coupling agent to covalently attach the coupling agent to the solid substrate to form a coupling agent modified solid substrate;
contacting the coupling agent modified solid substrate with the polymer of the formula (I) to covalently attach the polymer to the coupling agent modified solid substrate to form a polymer and coupling agent modified solid substrate; and
contacting the polymer and coupling agent modified solid substrate with a nucleic acid probe, optionally in the presence of a modulating small molecule, to covalently attach the nucleic acid probe to the polymer and coupling agent modified solid substrate to form the article, wherein the modulating small molecule can control the density of the nucleic acid probes attached to the polymer by using different ratio of the modulating small molecule to nucleic acid probes.

In embodiments, the modulating small molecule is an amine containing small molecule. The modulating small molecule can be selected, for example, from ethanolamine, and amine-terminated poly- or oligo-ethylene glycol. The modulating small molecule can also prevent non-specific binding of biomolecules to the surface during sequencing, and reduce the background signal over the sequencing cycle.

In embodiments, the method can further comprise controlling the density of the nucleic acid probes by determining and selecting, in advance, by for example, stoichiometry, the ratio of polymer to nucleic acid probes.

In embodiments, the disclosure provides a method of using the abovementioned article for nucleic acid sequence analysis, comprising:
contacting the article with a sample potentially containing one or more target nucleic acids having a complementary nucleic acid sequence to the nucleic acid probe.

The present disclosure is advantaged in several aspects, including for example:

The present invention enables rapid covalent coupling of amine-terminated nucleic acid (such as DNA) probe molecules (e.g., 5′-amine-dA30, or 5′-amine-dT30) to a solid support. The coupling can be completed, for example, within one hr, compared to a typical 16 hr coupling reaction when a bifunctional linker (e.g., BS3, bis(sulfosuccinimidyl)suberate) is selected to couple an amine-terminated DNA to an amine-presenting surface (e.g., ATPES coated surface).

The disclosure provides a more stable attachment of DNA probe molecules onto a solid support, compared to the coupling using a bifunctional linker or other means. This is mostly due to the multivalent, strong anchorage of the polymer layer to the amine presenting surface (e.g., APTES). This is significant, given that DNA sequencing often needs to run many reaction cycles, and involves some harsh treatments such as NaOH rinsing to denature any duplex DNA before sequencing read. In contrast, bifunctional linker-based attachment is mostly linear, that is, a surface structure of amine-bifunctional linker-DNA probe, which is prone to degradative loss or surface separation arising from these harsh chemical treatments.

The disclosure also provides a method of precise control of (and ultimately more efficient) hybridization between the probe DNA molecule and a target DNA fragment containing an adaptor sequence complementary to the probe. This is mostly due to the flexibility of the polymer chains even after coating. In contrast, for the bifunctional linker based DNA attachment, these DNA molecules are very close to the surface, thus preventing efficient hybridization.

The disclosure also provides precise control of the density of the DNA probe molecules attached to the surface, which permits user determined adjustability of the DNA hybridization efficiency, and subsequent clustering efficiency. This control can be achieved at either of two different chemical reactions. First, the amine reactive polymer is partially modified and derivatized with a small organic amine molecule (e.g., propylamine or the like). This controls the solubility of the polymer in solution for coating and the reactive sites once attached to the amine presenting surface. Second, the amine-terminated DNA probe molecules are incubated with the amine reactive polymer modified surface in the presence of a modulating small molecule, for example, ethanolamine or like agents, at a specific concentration. This step controls the covalent coupling reaction and, in turn, the density of the DNA probe molecules attached. The combination of these two different reactions permit excellent density control of the nucleic acid probe molecules or DNA probe molecules attached to the surface of the substrate, and ultimately the density of clusters formed, and the efficiency of the sequencing.

The present disclosure provides an article and method that is particularly useful for sequencing-by-synthesis based next generation sequencing (NGS) techniques, where polyclonal clusters are generally formed before sequencing. The cluster generation can be achieved using, for example, bridge amplification, exclusion amplification, or template walking approach, after the probe and DNA fragment molecules are attached to the surface.

The present disclosure is also applicable to NGS using nano-patterned flow cells. Nanopatterning can be achieved using state-of-the-art photolithography or nano-imprinting approaches.

The present disclosure is also useful for any biomolecular analysis using nucleic acid-based biosensors, where the density of the probe nucleic acid molecules attached is significant to the success of bioassays.

The present disclosure is also advantaged is several other aspects, including for example:

The disclosed flow cell device provides an amine reactive, polymer modified surface, where the amine reactive polymer is pre-derivatized with, for example, an amine containing small molecule to control the number and nature of the amine reactive sites. The pre-derivatized polymer can also have better solubility in solvents such as isopropyl alcohol, ethanol, or N-methyl-2-pyrrolidone (NMP), and can provide a uniform coating of the surface of a flow cell. At least one surface of the flow cell is pre-coated, i.e., reacted, with an amine containing silane prior to contacting the surface with the amine reactive, polymer.

The flow cell can include, for example, two solid substrates bound together using, e.g., a laser-assisted process, a tape, or a polyimide adhesive. The two substrates can be the same or different. The substrate can be, for example, plastic, glass, silicon, fused silica, or quartz. The flow cell defines a chamber or cavity. The flow cell can include, e.g., ports for media flow, e.g., liquid, into and out of the chamber (see Illumina.com; Illumina Sequencing Technology, Spotlight: Illumina® Sequencing).

The amine containing silane can be, for instance, mono-, di-, and tri-aminosilane, such as γ-aminopropylsilane, 3-(aminopropyl)triethoxysilane (APTES), 3-aminopropyl)trimethoxysilane, 3-aminopropyldimethylmethoxysilane, 3-aminopropyl(diethoxy)methylsilane, aminopropylsilsesquioxane (APS), N-[3-(trimethoxysilyl)propyl]ethylenediamine, or N1-(3-trimethoxysilylpropyl)diethylenetriamine.

The amine containing small molecule can be, for example, propylamine, allylamine, ethanolamine, or like molecules or amines.

The amine reactive polymer can be, for example, poly(ethylene-alt-maleic anhydride (EMA), styrene maleic anhydride (SMA), maleic anhydride copolymer such as poly(methyl vinyl ether-alt-maleic anhydride), and like polymers, or combinations thereof (see commonly owned U.S. Pat. No. 7,981,665).

In embodiments the present disclosure also provides a flow cell having a DNA probe molecule modified surface, where the density of the DNA probe molecules can be precisely controlled so that excellent DNA hybridization, subsequent polyclonal cluster formation, and sequencing can be achieved. The DNA probe molecule can be covalently attached to the amine reactive polymer coated flow cell surface by incubating the polymer surface modified flow cell surface with an amine terminated DNA probe molecule in the presence of a modulating second small molecule at a specific concentration.

The presence of the modulating second small molecule can be used to, e.g., control the degree of the coupling reaction of the amine terminated DNA probe molecule to the polymer surface, which can control or determine the density of the probe molecules attached. The ratio of the amine terminated DNA probe molecule to the modulating second small molecule determines the coupling degree of the probe DNA molecule. Preferably, the probe to the modulating small molecule mole ratio (P:S) can be, for example, 0.01:1, 0.1:1, 1:1, 1:2, 1:5, 1:10; 1:20, 1:50, 1:100, 1:1000, 1:10,000, and like ratios, including intermediate values and ranges, depending on the density desired. For instance, for random clustering using bridge amplification, the density of the DNA probe molecule is preferably relatively low (e.g., 10,000 per square micrometer of surface area). For random clustering using template walking the density of the DNA probe molecule is preferably relatively high (e.g., 250,000 per square micrometer of surface area).

The DNA probe molecule can be, for example, 5′-amine terminated dA30, 3′-amine terminated dA30, amine terminated dT30, and like probe molecules. In embodiments, the DNA probe molecule can be substituted with an RNA probe molecule for sequencing RNA.

In embodiments, the modulating second amine containing small molecule preferably is ethanolamine or amine terminated poly- or oligo-ethylene glycol. The reaction of ethanolamine with the anhydride groups of the polymer coating results in an OH-terminated, OH-rich surface, which can prevent non-specific binding, and provide a preferred low background signal.

In embodiments, the disclosure provides a flow cell having an array of discrete spots of amine reactive polymer coating, or covalently bound DNA probe molecules. The nano-patterning can be achieved by, for example, state-of-the-art photolithography or nano-imprinting techniques.

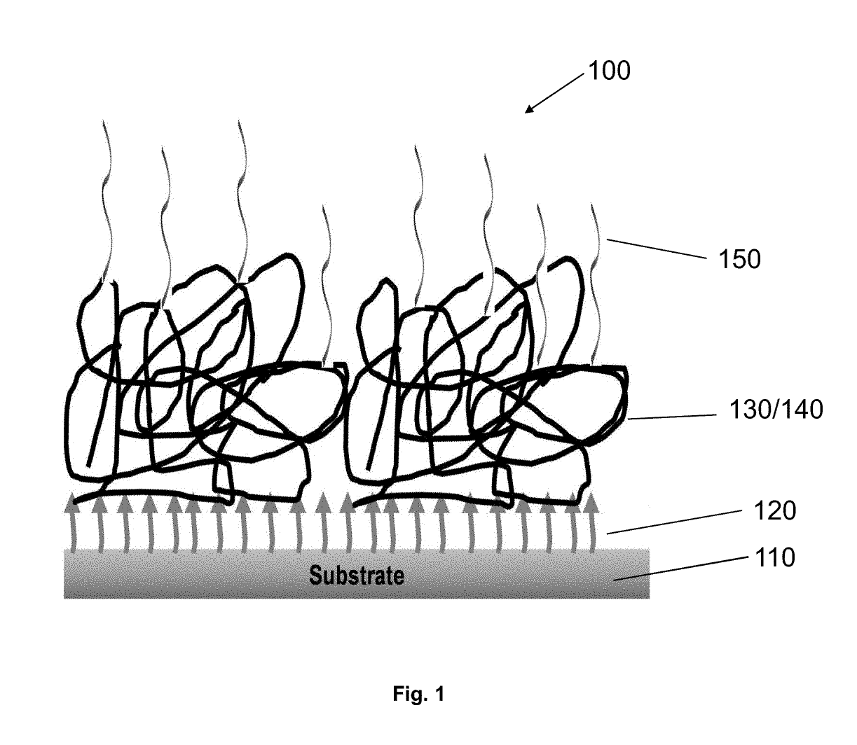

Referring to the Figures, FIG. 1 shows a schematic (100) of covalently coupled DNA probe molecules to a solid support. A solid support or substrate (110) is first modified with amine-presenting silane molecule (120), followed by covalently coupling of an amine reactive polymer (130) to form an amine reactive polymer coating or layer (140), and finally covalently coupling of an amine terminated DNA probe molecule (150).

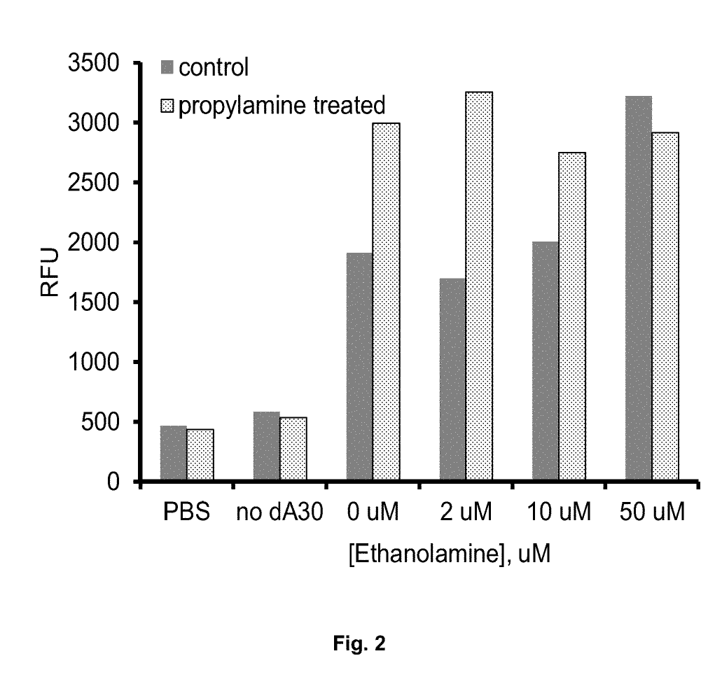

FIG. 2 shows a bar chart of the fluorescent intensity of a dA30-presenting surface after being hybridized with Cy3-labeled dT30. The dA30-presenting surface was made by first coating a glass substrate with γ-aminopropylsilane, followed by covalent attachment of non-derivatized (Control; solid bars) or propylamine-derivatized poly(ethylene-alt-maleic anhydride) (propylamine treated; dotted bars), and finally 5′-amine-dA30. The slide was scanned using a fluorescence scanner after incubating 1 microM Cy3-labeled dA30 for 45 min in the absence and presence of ethanolamine at three specific concentration (i.e., 2, 10, and 50 microM; as indicated in the graph) and rinsed three times using phosphate buffer. The surface treated with phosphate buffer (“PBS”), or the surface having “no dA30” after being incubated with Cy3-labeled dT30, was also examined and used as negative controls.

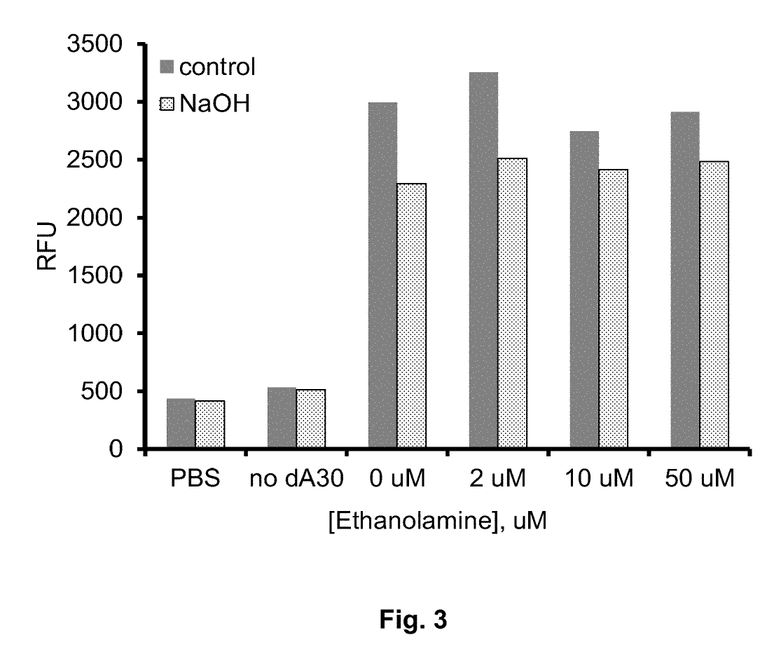

FIG. 3 shows a bar chart of fluorescent intensity of a dA30-presenting surface after being treated with 0.05M NaOH and subsequently hybridized with Cy3-labeled dT30. The dA30-presenting surface was made by first coating the glass substrate with γ-aminopropylsilane, followed by covalent attachment of propylamine-derivatized poly(ethylene-alt-maleic anhydride) and 5′-amine-dA30. Afterwards, the dA30 presenting surface was incubated with buffer (control; solid bars) or 0.05M NaOH (NaOH treated; dotted bars) for 5 min. After washing the surface, the surface was incubated with 1 microM Cy3-labeled dA30 for 45 min in the absence and presence of ethanolamine at three specific concentration (2, 10, and 50 microM; as indicated in the graph) and rinsed three times with phosphate buffer. The slide was scanned using a fluorescence scanner. The surface treated with phosphate buffer (“PBS”), and the surface having “no dA30” (PBS), after incubated with Cy3-labeled dT30, were also examined and used as negative controls.

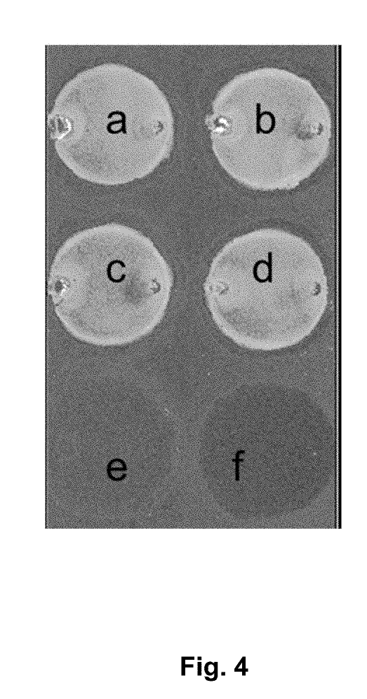

FIG. 4 shows a fluorescent image (original color image available; not provided) of a well plate surface after being hybridized with Cy3-labeled dT30. The slide was first coated with γ-aminopropylsilane, followed by covalent attachment of propylamine-derivatized poly(ethylene-alt-maleic anhydride). The slide regions were separately treated, as follows where each letter corresponding to images of the well plate wells labelled a, b, c, d, e, and f:

(a) The surface was incubated with 10 microM amine-dA30 for 45 min, rinsed three times, and finally hybridized with 1 microM Cy3-labeled dT30 target.
(b) The surface was incubated with 10 microM amine-dA30 in the presence of 2 microM ethanolamine for 45 min, rinsed three times, and finally hybridized with 1 microM Cy3-labeled dT30 target.
(c) The surface was incubated with 10 microM amine-dA30 in the presence of 10 microM ethanolamine for 45 min, rinsed three times, and finally hybridized with 1 microM Cy3-labeled dT30 target.
(d) The surface was incubated with 10 microM amine-dA30 in the presence of 50 microM ethanolamine for 45 min, rinsed three times, and finally hybridized with 1 microM Cy3-labeled dT30 target.
(e) The surface was directly incubated with 1 microM Cy3-labeled dT30 target.
(f) The surface was directly incubated with phosphate buffer.
Finally, the slide was rinsed, dried, and fluorescently scanned.

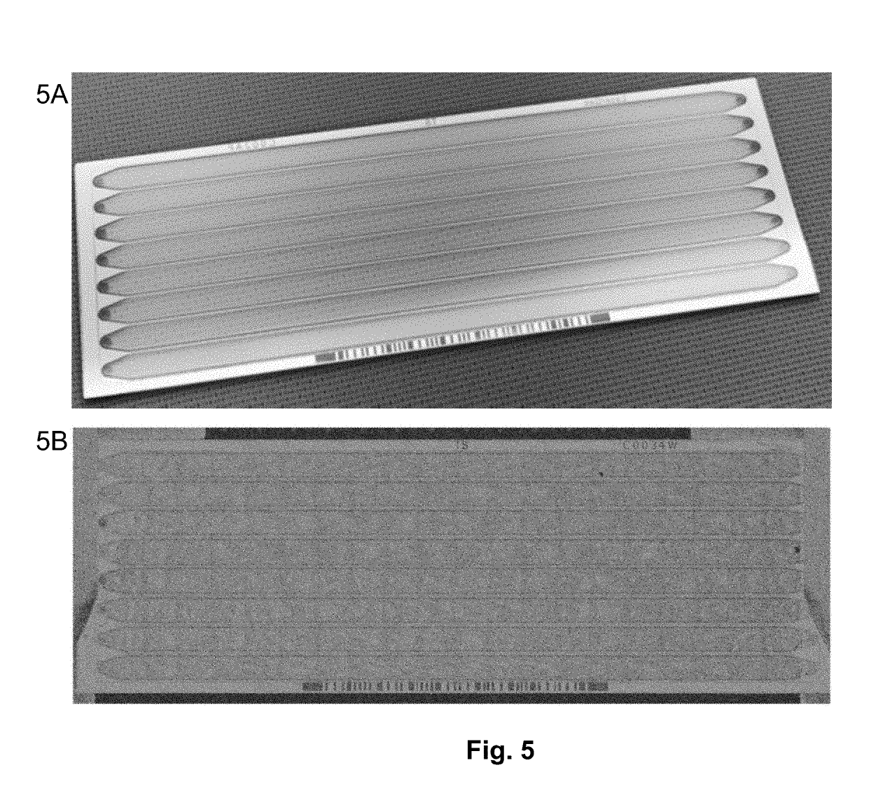

Referring again to the Figures, FIG. 5A shows a photo image of an entire flow cell, and FIG. 5B shows a confocal fluorescent image of an entire flow cell having 8 channels, each consisting of amine-terminated dA30 attached to a reactive polymer coating, after being hybridized with Cy3-labeled dT30. Specifically, all channels are first coated with γ (gamma)-aminopropylsilane, followed by covalent attachment of propylamine-derivatized poly(ethylene-alt-maleic anhydride). Afterwards, all channels were incubated with 50 microM 5′-amine-terminated dA30 in the presence of 100 microM ethanolamine, followed by further treatment as mentioned below. Channel 1 to 8 from top to bottom are:

Channel 1, 2, 7, and 8: rinsed with phosphate buffer (PBS) three times.

Channel 3, and 6: incubated with 0.05 M NaOH 5 min, rinsed with PBS three times.

Channel 4, and 5: rinsed with 60° C. water five times, 1 min each.

Finally, all channels were incubated with 1 microM Cy3-labeled dT30 for 45 min. After rinsing with PBS three times and dried, confocal microscopy was used to scan the entire flow cell. All fluorescence images collected were assembled together to form the fluorescent image of the entire flow cell as shown.

### EXAMPLES

The following Examples demonstrate making, use, and analysis of the disclosed article and methods in accordance with the above general procedures.

### Example 1

Silane coating Glass slides were coated with 3-(aminopropyl)triethoxysilane (APTES) using a chemical vapor deposition protocol with a YES silane coater.

Glass slides were alternatively coated with 5% aminopropylsilsesquioxane (APS) in water, followed by rinsing and drying under nitrogen. Our results showed that either surface modification coating results in a similar: polymer coating of formula (I); dA30 attachment; and subsequent Cy3-labeled dT30 hybridization efficiency.

### Example 2

EMA coating The APS coated slides were further used to covalently couple EMA with or without pre-derivatization with propylamine. Specifically, poly(ethylene-alt-maleic anhydride) (EMA), as received from commercial vendors, was first dried under argon, followed by dissolving into anhydrous N-methyl-2-pyrrolidone (NMP) in the absence or the presence of propylamine at a specific concentration to form stock solutions of EMA or derivatized EMA (dEMA), respectively. The coating was performed by incubating the silane coated glass slides of Example 1 with an EMA solution in NMP or a dEMA solution in NMP for 30 min, followed by rinsing, drying under argon, and packaging in a plastic mailer under nitrogen. Results showed that the EMA coated slides have higher hydrophobicity compared to the dEMA coated slides.

### Example 3

Covalent attachment of amine terminated dA30 The EMA or dEMA surface modified slides of Example 2 were further used to covalently couple an amine terminated dA30 by incubating either of the EMA or dEMA coated the slides with 10 microM 5′-amine-dA30 (purified) in phosphate-buffered saline (PBS) for different times in the absence and presence of ethanolamine at different concentrations. After rinsing with PBS three times and drying, the dA30 coated slides were stored under nitrogen or used directly for dT30 hybridization.

### Example 4

Hybridization with Cy3-labeled dT30 The dA30 coated slides of Example 3 were used to hybridize with 1 microM Cy3-labeled dT30 in PBS for 30 min. After hybridization, the slides were washed with PBS three times, dried, and scanned using a GenePix fluorescent scanner. The results showed that the dA30 absent surface gave rise to low background, similar to bare glass or the dA30 coated surface that was incubated with the buffer only, suggesting little or low non-specific binding of fluorescent dT30 (see FIGS. 2 and 3). Our results also showed that the co-presence of ethanolamine during the dA30 coupling step impacted the fluorescence arising from the hybridization with Cy3-labeled dT30 (FIG. 2). Interestingly, on the EMA coated slide, as the concentration of ethanolamine increased, the fluorescence intensity increased, suggesting that in the absence or the presence of low concentration of ethanolamine, the density of dA30 attached is high such that after hybridization they are self-quenching in fluorescence due to closely packed Cy3-labeled dT30 after hybridization.

In contrast, for one the dEMA coated slides, the fluorescence intensity arising from the Cy3-labeled dT30 hybridization seems insensitive to the presence of ethanolamine, regardless of its concentration. This result suggests that there is little in fluorescence self-quenching, due to the relatively low density of dA30 attached, which in turn, resulted in less hybridization with Cy3-labeled dT30.

We also examined the stability of dA30 attachment by incubating the dA30 surface modified slides with 0.05 M NaOH. NaOH treatment is known to cause the breakage of the Si—O—Si bonds, resulting in the loss of attached molecules. Results showed that NaOH treatment only slightly decreased the fluorescence intensity arising from the hybridization of Cy3-labeled dT30 (FIG. 3), suggesting that the dA30 attachment is stable.

Fluorescence images obtained using the scanner suggest that the fluorescence of a spot is quite uniform (FIG. 4).

### Example 5

DNA hybridization in a flow cell A glass substrate was first chemically etched to form eight individual channels. After rinsing with water, the substrate was coated with a 5 wt % APS solution and then rinsed three times and dried. The APS coated substrate was further coated with derivatized EMA. After rinsing and drying, the substrate was bound to a cover glass to form a flow cell using a laser assisted process such that there are eight channels formed between the glass substrate and the cover glass, each channel has an inlet and an outlet (FIG. 5A). After the flow cell was assembled, all channels were incubated with 25 microliters of a PBS buffered solution containing 50 microM 5′-amine-terminated dA30 and 100 microM ethanolamine for 1 hr. Afterwards, channels 3 and 6 were further treated with 0.05M NaOH for 5 min, while channels 4 and 5 were further treated with 60° C. hot water five times, each for 1 min. Finally, all channels were rinsed with PBS buffer three times, and dried. The entire flow cell was scan imaged with a confocal microscopy. The fluorescence image of the entire flow cell, as shown in FIG. 5B, showed that the fluorescence intensity is very uniform within each channel and across all channels, suggesting that the NaOH or hot water treatment had little impact on the dA30 attached and the dA30 attached is very stable and supports efficient hybridization of dT30.

The disclosure has been described with reference to various specific embodiments and techniques. However, it should be understood that many variations and modifications are possible while remaining within the scope of the disclosure.

What is claimed is:

1. A flow cell article comprising:
a chamber; and
at least one surface of the chamber comprising:
a solid substrate having a reactive surface comprising:
a coupling agent covalently attached to the solid substrate;
a polymer of the formula (I) covalently attached to the coupling agent; 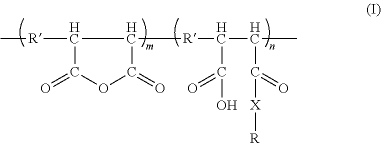
the polymer having at least one of: a plurality of maleic anhydride reactive groups (m), a plurality of reacted groups (n), or a mixture of (m) and (n), where
X is a divalent NH, O, or S;
R is H, a substituted or an unsubstituted, linear or branched alkyl group, an oligo(ethylene oxide), an oligo(ethylene glycol), or a dialkyl amine;
R′ is a residue of a first unsaturated monomer that has been copolymerized with maleic anhydride; the relative ratio (m:n) of the maleic anhydride reactive groups to the reacted groups is from 0.5 to 10;
m is of from 1 to 10,000 and n is of from 0 to 9,500; and
a nucleic acid probe covalently attached to the polymer.

2. The article of claim 1, wherein the nucleic acid probe is an amine-terminated nucleic acid, or a mixture of amine-terminated nucleic acids thereof.

3. The article of claim 1, wherein the solid substrate is a glass, a glass ceramic, a silicon, a fused silica, or quartz.

4. The article of claim 1, wherein the nucleic acid probe molecule has a density of 1 to 500,000 probe molecules per square micrometer of surface area.

5. The article of claim 1, wherein the coupling agent is a silane, silsesquioxane, or a mixture thereof.

6. The article of claim 5, wherein the silane is 3-(aminopropyl)triethoxysilane, and the silsesquioxane is aminopropylsilsesquioxane.

7. A method of making the article of claim 1, comprising:
contacting a solid substrate with a coupling agent to covalently attach the coupling agent to the solid substrate to form a coupling agent modified solid substrate;
contacting the coupling agent modified solid substrate with the polymer of the formula (I) to covalently attach the polymer to the coupling agent modified solid substrate to form a polymer and coupling agent modified solid substrate; and
contacting the polymer and coupling agent modified solid substrate with the nucleic acid probe to covalently attach the nucleic acid probe to the polymer and coupling agent modified solid substrate to form the article.

8. The method of claim 7, wherein the solid substrate is a glass, a glass ceramic, a silicon, a fused silica, or quartz.

9. The method of claim 7, further comprising controlling the density of the nucleic acid probes by selecting the ratio of polymer to nucleic acid probes.

10. The method of claim 7, further comprising a modulating small molecule in the contacting the polymer and coupling agent modified solid substrate with the nucleic acid probe step.

11. The method of claim 10, wherein the modulating small molecule is ethanolamine, an oligo-ethylene glycol, a poly-ethylene glycol, or a mixture thereof.

12. The method of claim 10, wherein the modulating small molecule controls the density of the nucleic acid probes attached to the polymer by using different ratios of the modulating small molecule to nucleic acid probes.

13. A method of using the article of claim 1 for nucleic acid sequence analysis, comprising:
contacting the article with a sample potentially containing one or more nucleic acids having a complementary nucleic acid sequence to the nucleic acid probe.
